(19) United States
(12) Patent Application Publication
(10) Pub. No.: US 2005/0234299 A1
(43) Pub. Date: Oct. 20, 2005

(54) Proctoscope

(76) Inventors: Jurgen Eitenmuller, Castrop-Rauxel (DE); Harald Brielmayer-Pomino, Markdorf (DE)

Correspondence Address: BACHMAN & LAPOINTE, P.C., 900 CHAPEL STREET, SUITE 1201, NEW HAVEN, CT, 06510, US

(21) Appl. No.: 10/520,235

(22) PCT Filed: Jul. 8, 2003
(86) PCT No.: PCT/EP03/07291

§ 371 Date: Jan. 4, 2005

(30) Foreign Application Priority Data
Jul. 9, 2002 (DE) 102 31 004.1

(51) Int. Cl. A61B 1/06

(52) U.S. Cl. 600/160; 600/138; 600/129; 600/179

(57) ABSTRACT

A proctoscope which comprises a body preferably in the form of a hollow cylinder which makes it possible to insert it in the anal canal of a patient. The body of the proctoscope is provided with a construction as a level of the part of a distal end and with an aperture and a handhold on the other end. The body of the proctoscope is also provided with a passage in order to form a surgical window oriented towards the internal free zone thereof. The surgical window is formed in the external wall of the body in such a way that it can enter the part of the distal end by contracting itself.

### BACKGROUND OF THE INVENTION

The present invention relates to a proctoscope for the diagnostic and therapeutic treatment of hemorrhoids.

It is estimated that in Western industrial countries, based on 100,000 people, approximately 1000 doctors' appointments are required each year in order to diagnose and treat problems of the hemorrhoidal arteries. Depending on the severity of these problems (the medical literature has divided hemorrhoidal disorders into four different stages), various treatment methods exist for treating the individual stages. It is customary, in particular for the advanced stages of two or above, for the patients to be treated under anesthetic, sometimes in conjunction with several days' stay in hospital.

Since about the middle of the 1990s, a less invasive method which is potentially also suitable for the out-patient treatment of hemorrhoidal disorders has become known, originating in Japan—the so-called H.A.L. method (Hemorrhoidal Artery Ligation). In this H.A.L. method, a special proctoscope is inserted into a patient's anus and the hemorrhoidal arteries are detected by means of ultrasound Doppler sonography, which makes it possible to place the proctoscope with respect to the hemorrhoid. Through an operation window of the proctoscope, the operator can then suture and ligate the relevant vessels.

This leads to a reduction in the blood supply to the hemorrhoids, the pressure in the vessels decreases, and after a short time the hemorrhoids shrink. This known intervention is advantageous in that it is normally carried out as an out-patient procedure and without any anesthesia, and has an extremely high success rate.

Proctoscopes of the generic type, as required for carrying out the H.A.L. method, are sold in Germany for example by the company AMI. These proctoscopes allow the operator to make a reliable diagnosis visually and/or by means of Doppler sonography, and the operation window makes it possible to carry out the necessary operative steps to treat the hemorrhoidal problem.

In such generic devices, however, the operation window is formed in a side wall or casing portion of the cylindrical proctoscope body. When the device is inserted into a patient's anus, this then leads to the serious disadvantage that the artery which (depending on the stage) is already inflamed or wounded rubs over the (sharp) edge of the operation window formed as a cut-out in the hollow cylinder, and further adjustment or positioning movements of the proctoscope body by the operator further aggravate this situation. As a result, this means that the generic devices can be used to carry out the advanced H.A.L. method usually only in the case of relatively minor hemorrhoidal disorders, and particularly severe stages have to be operated on in the conventional manner with the abovementioned disadvantages.

It is therefore an object of the present invention to improve a generic proctoscope designed for carrying out the H.A.L. method such that it can be used in a simpler and, from the patient's point of view, less painful manner and for a greater number of possible applications, and makes it possible to avoid or reduce the conventional need to administer drugs.

### SUMMARY OF THE INVENTION

The foregoing object is achieved by providing a proctoscope comprising a proctoscope body which is preferably hollow-cylindrical and is designed to be inserted into a patient's anus, said proctoscope body having a tapering at a distal end portion, an opening at the other end and also a handle portion, wherein the proctoscope body has a hole designed to form an operation window toward a free interior space of the proctoscope body, and means for illuminating the operation window and/or means for detecting vessels by means of sensors, in particular by means of ultrasound, may be provided in the proctoscope body, characterized in that the operation window is formed in the outer wall of the proctoscope body such that it extends into the tapering distal end portion.

In an advantageous manner according to the invention, at the distal end portion (i.e. the insertion end of the proctoscope body), the hole which forms the operation window is formed such that it extends, preferably as a cut-out in the hollow-cylindrical proctoscope body or the conically tapering distal end portion, into the tapering region. Not only is the size of the operation window thus increased in comparison to known devices but also, with respect to a maximum diameter of the proctoscope body, a front (insertion) edge of the operation window is smaller than the maximum diameter, which on the one hand facilitates insertion of the proctoscope body and on the other hand at the same time largely reduces further irritation of the relevant hemorrhoidal artery. In particular, the disadvantageous scraping of the tissue over the edge of the operation window, as occurs in generic known devices, is thereby less severe.

If then, as provided in one further development of the invention, the tapering distal end portion is at least partially formed of a solid material, e.g. plastic, the hemorrhoidal artery or the intestinal mucosa remains virtually unaffected by the insertion of the proctoscope and any further adjustment movements. Besides positive effects of the reduction in pain (and the avoidance of anesthesia which would otherwise be required), the device according to the invention is thus suitable even for treating hemorrhoidal problems in advanced stages, for example in the case of strangulated, thrombosed or fibrosed nodes, successfully and without requiring that the patient be kept in hospital. As a result, the present invention considerably expands the application possibilities of the H.A.L. method for treating hemorrhoids.

According to one advantageous further development of the invention, it is provided that the proctoscope body cooperates with a push-in obturator which in particular has the function, when it is pushed in, of closing off the operation window on the inside and particularly preferably completely, in order then to be pulled out once the proctoscope body is inserted so that the operation window is freely accessible to the operator for further operation steps. The disadvantageous effect of operation window edges (which are still present) on the relevant blood vessel is hereby further reduced, or even completely prevented if the casing is appropriately rounded and the operation window is completely closed.

In order to simplify handling of this obturator, the latter has at one end a handle portion which protrudes out in the inserted state and at the other end it is provided with a suitably tongue-shaped extension for reliably closing off the operation window.

Particular preference is given to providing an ultrasound Doppler probe permanently on or in the proctoscope body, suitably adjacent to the operation window. By virtue of the permanent installation, preferably permanently integrated in the wall of the proctoscope body, cleaning problems can be overcome in a simple manner and, by means of the handle portion preferably designed as an angled grip, the ultrasound probe can then be easily pushed into its desired position or pivoted by the operator; the Doppler electronics, which are known per se and are connected to the ultrasound probe (it is particularly preferable to pass the corresponding supply lines through the appropriately hollowed-out handle portion), then allow simple reporting by signals as to the guidance and handling of the proctoscope.

While one particularly preferred embodiment of the invention provides for use of an ultrasound Doppler probe, the invention equally includes the use of other methods, preferably imaging methods, for detecting the blood vessel, in particular according to the so-called duplex method.

Moreover, it is particularly preferable within the scope of the present invention to make it easier for the operator to carry out a visual inspection. For this purpose, according to one development lighting means are provided in the interior space of the proctoscope body, wherein said lighting means may be provided either (seen from the opening or observation direction of the observer) in front of or behind the operation window. In addition or as an alternative, mirrors or other reflection means may be provided which make it easier for the operator to see the operation window (and the intervention site), even more preferably in the form of a backlit mirror.

As a result, the present invention makes it possible to use the advanced H.A.L. method to treat hemorrhoids in a considerably more flexible manner and for a greater number of applications, wherein the discomfort of the patient is considerably reduced compared to generic known devices for an H.A.L. method and at the same time the operating comfort and flexibility in terms of operation can be greatly improved for the operator.

### BRIEF DESCRIPTION OF THE DRAWINGS

Further advantages, features and details of the invention emerge from the following description of preferred examples of embodiments and with reference to the drawings, in which:

FIG. 1 shows a side view of the proctoscope according to a first preferred embodiment of the present invention;

FIG. 2 shows a longitudinal section through the arrangement of FIG. 1;

FIGS. 3, 4 respectively show a side view of and longitudinal section through an obturator for use with the arrangement of FIG. 1 and FIG. 2;

FIG. 5 shows a front view from the side of the closure portion on the obturator of FIG. 4 and

FIG. 6 shows a sectioned side view through the arrangement of FIG. 2 with the obturator of FIG. 3 pushed in.

### DETAILED DESCRIPTION

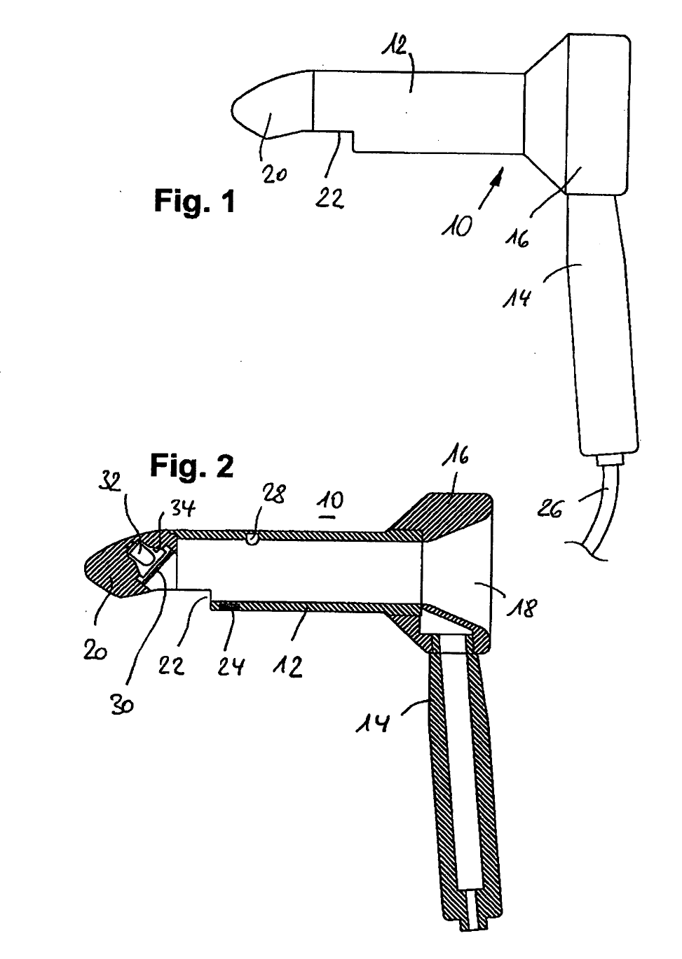

The proctoscope of FIG. 1 and FIG. 2 consists, as can be seen particularly clearly from the sectional view, of a proctoscope body 10 made of a suitable plastic material (typically PPSU) with a handle portion 14 which projects at right angles to a hollow-cylindrical central portion 12 of the proctoscope body 10. Central portion 12 and handle portion 14 are joined by a transition or opening portion 16 which (cf. FIG. 2) conically widens a constant cylindrical inner width of the central portion 12 (typical inner diameter 20 to 22 mm) and thus forms at the end an intervention or observation opening 18.

At the other end the central portion 12 has, at the so-called insertion or distal end, a tapering proctoscope tip 20 which is essentially formed of solid plastic material.

As can be seen in FIGS. 1 and 2, in the transition region between the conically tapering proctoscope tip 20 and the central portion 12, an operation window 22 is formed by making a cut-out in the hollow-cylindrical central portion 12 (in the cross-sectional direction). More specifically, this cut-out which forms the operation window 22 is made in such a way that it extends over its main region essentially parallel to the axis of symmetry or longitudinal axis through the central portion 12, and only in the front region toward the proctoscope tip 20 does it describe a slight angle bent outward.

In this way, a hole is made in the central portion 12 and the proctoscope tip 20, said hole having an effective cross-sectional area of between 350 and 400 mm², this being a significant increase compared to operation windows of conventional devices (which are formed only in the cylindrical casing walls of a central portion). Moreover, the partially solid design of the proctoscope tip 20 with the plastic material ensures that an operation opening is produced, in particular at the critical insertion side, which is easy to clean, mechanically stable and gives the patient minimum discomfort.

FIG. 2 additionally shows, in the sectional view from the side, an ultrasound sensor unit 24 which is provided directly adjacent to the operation window 22 in the direction toward the grip 14, specifically such that the crystal element of the sensor is permanently surrounded by the plastic material of the proctoscope body 10. Shown only schematically is a supply line 26 (FIG. 1) which takes the ultrasound signals to and from an externally provided ultrasound Doppler unit (known per se) for vessel diagnosis, said signals being carried within the proctoscope via connections (not shown in more detail).

The supply line 26 also supplies lighting means provided within the proctoscope body 10, on the one hand a proximal lighting means 28 provided to the right above the operation window 22 and on the other hand a distal lighting means accommodated in the rear region of the proctoscope tip 20 behind an angled glass element 30, said distal lighting means additionally being surrounded by a schematically shown reflector element 34.

By virtue of its positioning at an angle above the operation window 22, the glass element 30 firstly ensures that an observer looking into the proctoscope through the opening portion 16 can see the operation window or a body surface exposed therein; on the other hand, the element 30 is designed such that luminous radiation of the second distal lighting means 32 can pass unhindered through said element and reach the operation window 22.

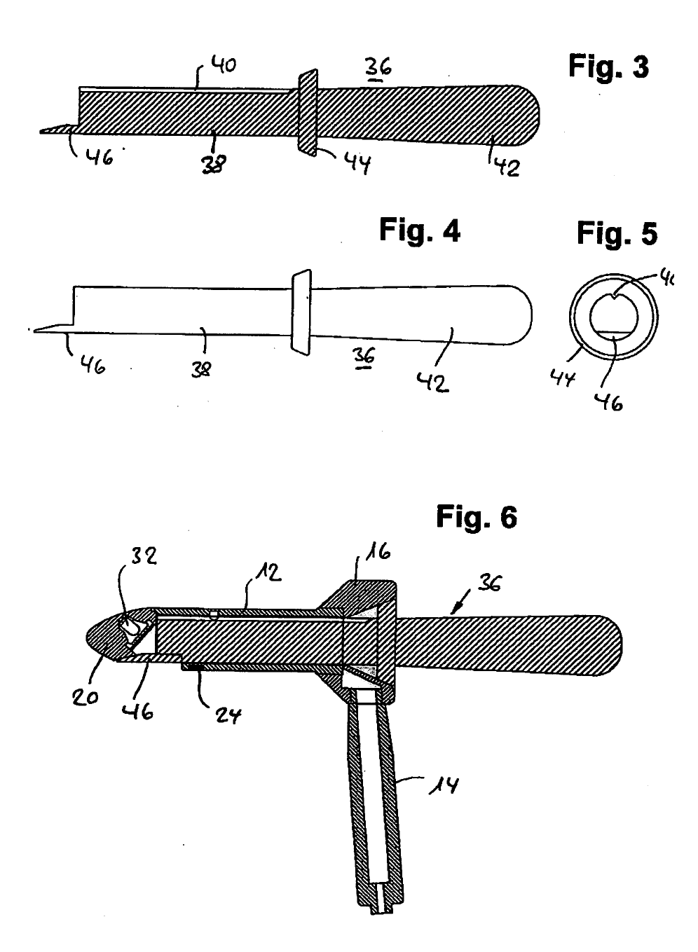

FIGS. 3 to 5 show an obturator element 36 as an important accessory part for the arrangement of FIG. 1 and FIG. 2; FIG. 6 shows the units in the pushed-in (assembled) state.

A plastic obturator 36 made of an appropriate material suitable for medical purposes (e.g. PPSU) has a solid-cylindrical portion 38 which is adapted to the inner diameter of the central portion 12. Formed in the upper region (cf. the front view of FIG. 5) is a longitudinal groove 40 which extends along the portion 38 and which—as a dual function—on the one hand accommodates and guides the slightly protruding proximal lighting means 28 and on the other hand ensures that, as the obturator element 36 is guided into and out of the proctoscope body 10, no air is introduced into or taken out of the interior space, and thus no irritation to the patient's body exposed through the operation window 22 is caused on account of a negative or excess pressure.

At one end of the cylindrical portion 38, the obturator element 36 has a handle portion 42 which (cf. the completely pushed-in state shown in FIG. 6) additionally forms a conically tapering stop element 44 for a fixed seat in the opening portion 16 and projects backward from the overall arrangement.

At the other end, a closure portion 46 is integrally formed in one piece on the portion 38 in the manner of a tongue-like protrusion 46, which closure portion (cf. the sectional view in FIG. 6) completely and tightly closes off the operation window 22 in the pushed-in state of the obturator element 36 and additionally ensures a step-less, smooth transition between the outer surface and the proctoscope tip 20. For this purpose, in a manner corresponding to the tapering of the proctoscope tip 20, the closure element 46 is designed to taper slightly at the end.

For use, in the normal case the operator fits the proctoscope shown in FIG. 1 and FIG. 2 together with the obturator element shown in FIG. 3 and FIG. 4, and thus obtains the arrangement of FIG. 6. The operation window 22—which is typically oval—is completely closed, and the transition to the proctoscope tip 20 is step-less. This arrangement is then inserted by the operator into the patient's anus, and in the process the hemorrhoidal artery slides along the proctoscope tip 20 to the closure element 46 without any complications. Correct positioning takes place by means of the signals generated by the ultrasound sensor unit 24 (and sensor electronics connected downstream). The handle portion 14 also serves to orient the operator and for external observation, said handle portion, with respect to the position of the ultrasound sensor unit 24, being aligned with the latter and thus indicating the position thereof in the circumferential direction. At the operation site, i.e. in the desired position of the overall arrangement shown in FIG. 6, the operator then pulls the obturator element 36 out of the arrangement, and the operation window 22 is opened. Using operation techniques known per se from the H.A.L. method, the operator can then treat the relevant blood vessel in an appropriate manner; the lighting means 28, 32 which can be adjusted in terms of light intensity provide him with ideal observation conditions.

The present invention is not restricted to the example of embodiment described above. For instance it is possible in particular to form the proctoscope of any suitable material and in any dimensions, in particular including with respect to the specific configuration of the operation window, wherein the dimensions shown—length and width of the operation window approximately 30×24 mm, cut-out depth of the operation window with respect to the circumferential diameter approximately 26 mm—have been found to be advantageous.

While it was preferred within the context of the described example of embodiment to provide an ultrasound sensor unit, as an alternative it is advantageous and preferable to provide a different (imaging) sensor unit, in particular as a unit operating in accordance with the duplex method.

With regard to the ultrasound electronics assigned to the permanently installed sensor element 24, all possibilities for ultrasound diagnosis may be used, as are known per se and sold in particular including by the applicant.

The present invention thus provides an instrument by virtue of which the advantageous operation technique of Hemorrhoidal Artery Ligation can be extended to additional further fields of application, as a result of which both the flexibility of use of this patient-friendly, non-invasive method and the discomfort to the patient himself can be considerably reduced.

1. A proctoscope comprising a proctoscope body (12, 16, 20) which is hollow-cylindrical and is to be inserted into a patient's anus, said proctoscope body having a tapering at a distal end portion, an opening (18) at the other end and a handle portion (14), wherein the proctoscope body has a hole to form an operation window (22) toward a free interior space of the proctoscope body, means (28, 32) for illuminating the operation window and/or means (24) for detecting vessels by means of sensors are provided in the proctoscope body, wherein the operation window (22) is formed in an outer wall of the proctoscope body such that it extends into the tapering distal end portion (20).

2. The device as claimed in claim 1, further including a manually operable obturator (36) for insertable cooperation with the proctoscope body and is adapted to the free interior space such that, when the obturator is inserted into the proctoscope body through the opening (18), a closure portion (46) of the obturator at least partially closes off the operation window (22).

3. The device as claimed in claim 2, wherein the obturator has a handle portion (42) which in the inserted state protrudes out of the opening, and the closure portion (46) is designed as a tongue-like extension of an essentially cylindrical obturator casing (38).

4. The device as claimed in claim 1, wherein the operation window (22) is formed as a cut-out in the hollow-cylindrical proctoscope body and in the tapering distal end portion.

5. The device as claimed in claim 1, wherein the operation window opens up a free hole area of 350 to 400 mm² in the outer wall of the proctoscope body.

6. The device as claimed in claim 1, wherein the sensor comprises a duplex or ultrasound sensor (24) which is permanently installed in a wall portion of the proctoscope body adjacent to the operation window, wherein said sensor is connected to vessel detection electronics provided outside the casing of the proctoscope body.

7. The device as claimed in claim 1, wherein the handle portion is designed as a grip (14) at the opening end of the proctoscope body, said grip being angled with respect to the proctoscope body and forming a hollow space for the passage of supply lines (26) for the illuminating and/or vessel detection means and further indicating a circumferential position of the vessel detection means on the proctoscope body.

8. The device as claimed in claim 1, wherein the means for illuminating comprises lighting means (32) directed onto the operation window provided in the tapering end portion.

9. The device as claimed in claim 8, wherein a mirror and/or reflector element (30) is provided in the tapering end portion such that an observer can see the operation window (22) from the opening (18).

10. The device as claimed in claim 9, wherein the mirror and/or reflector element (30) is light-permeable to the lighting means (32) provided behind it.

11. A method of operating the proctoscope as claimed in claim 1, including the steps of:
closing the operation window in the proctoscope body;
inserting the proctoscope body into a patient's anus;
detecting blood vessels by means of ultrasound Doppler sonography and orienting the proctoscope body in reaction to the detection; and
opening the operation window.
